(19) United States
(12) Patent Application Publication
(10) Pub. No.: US 2026/0120887 A1
(43) Pub. Date: Apr. 30, 2026

(54) ACTIVE DETECTION SYSTEM FOR ADVERSE DRUG REACTIONS USING PERIODIC CONVERSION DATABASE AND ARTIFICIAL INTELLIGENCE

(71) Applicant: EVIDNET, INC., Seoul (KR)

(72) Inventors: Eunchan JANG, Gyeonggi-do (KR); Hyunki WOO, Gyeonggi-do (KR); Chanjung LEE, Seoul (KR)

(73) Assignee: EVIDNET, INC., Seoul (KR)

(21) Appl. No.: 19/488,731

(22) PCT Filed: Feb. 29, 2024
(86) PCT No.: PCT/KR2024/002623

§ 371 Date: Nov. 28, 2025

(30) Foreign Application Priority Data
May 30, 2023 (KR) 10-2023-0068851

(51) Int. Cl. G16H 50/30; G16H 10/60

(52) U.S. Cl. CPC G16H 50/30; G16H 10/60

(57) ABSTRACT

A method for generating a prediction result for an adverse drug reaction by utilizing artificial intelligence technology and performed by a computing device is disclosed. The method may include: building a periodically updated transformed database that is scheduled to be updated at predetermined time intervals by using raw data for detecting an adverse drug reaction; determining a plurality of input variables for training an artificial intelligence-based model for detecting the adverse drug reaction, based on data stored in the periodically updated transformed database; and training the artificial intelligence-based prediction model such that, in response to receiving the determined input variables, the prediction model outputs adverse reaction information for a specific drug and a specific disease, by using the determined input variables and a training dataset including adverse reaction information for the specific drug and the specific disease. A representative drawing may be FIG. 3.

### TECHNICAL FIELD

The present disclosure relates to an artificial intelligence field, and more particularly, to a method and a device for active detection of an adverse drug reaction utilizing an artificial intelligence technology.

### BACKGROUND ART

Drug adverse reactions are defined as harmful and unintended reactions to pharmaceutical products, and pose risks to patient health, ranging in severity from mild to very serious. In recent years, as drug sales have increased worldwide, drug adverse reactions have also been increasing. Accordingly, opinions are emerging that countermeasures are needed, and pharmaceutical safety evaluation and domestic and international regulatory agencies are researching the causes of drug adverse reactions and proactive response measures. The World Health Organization (WHO) manages VigiBase (https://who-umc.org/vigibase/), a global drug safety information database that collects information on drug adverse reactions occurring worldwide. The U.S.

Food and Drug Administration (FDA), as a regulatory agency for ensuring the safety and efficacy of pharmaceuticals, operates a database called FDA Adverse Event Reporting System (FAERS) to collect information on drug adverse reactions reported to the FDA. Side Effect Resource (SIDER) (http://sideeffects.embl.de/about/), managed by researchers at the University of Copenhagen in Denmark, is a database containing marketed drugs and their adverse reaction information. In Korea, the Ministry of Food and Drug Safety operates KAERS (https://kaers.drugsafe.or.kr/). From these various databases related to adverse drug reactions, standardized information on adverse drug reactions can be collected.

Recent research in the field of adverse drug reactions has been conducted utilizing artificial intelligence, with the primarily employed technologies including machine learning, deep learning, and natural language processing. According to a study that searched papers published from Jan. 1, 2015 to Jul. 9, 2021 and reviewed, among key terms related to artificial intelligence and drug safety, terms contained in their titles or abstracts, main application areas among retrieved papers were: identification of adverse events and drug reactions (57.6%), safety report processing (21.2%), drug-drug interaction extraction (7.6%), identification of populations at high risk of drug toxicity or personalized treatment guidelines (7.6%), adverse event prediction (3.0%), clinical trial simulation (1.5%), and linkage of predictive uncertainty in diagnostic classifiers, with the utilization of artificial intelligence in the field of adverse event and drug reaction prediction expected to further increase.

To actively detect adverse drug reactions, drug administration information and responses to administration (diagnostic test data, disease diagnosis data, nursing records, etc.) are essential. A database converted to common data model specifications using electronic medical records can collect data from multiple healthcare institutions and can more efficiently utilize data from healthcare institutions by employing a regularly updated database.

In this regard, Korean Patent Unexamined Publication No. 2008-0042256 is contrived.

### DISCLOSURE

### Technical Problem

The present disclosure is contrived in response to the above-described background art, and has been made in an effort to provide a method and a device for active detection of an adverse drug reaction utilizing an artificial intelligence technology.

The technical problems to be solved by the present disclosure are not limited to those mentioned above, and other technical problems not explicitly described herein will be clearly understood by those skilled in the art from the following description.

### Technical Solution

According to one embodiment of the present disclosure, a method for generating a prediction result for an adverse drug reaction using artificial intelligence technology performed by a computing device is disclosed. The method may include: building a periodically updated transformed database scheduled to be updated at predetermined time intervals using raw data for detecting an adverse drug reaction; determining a plurality of input variables for training an artificial intelligence-based prediction model for detecting the adverse drug reaction based on data stored in the periodically updated transformed database; and training the prediction model such that, in response to receiving the determined input variables, the prediction model outputs adverse reaction information for a specific drug and a specific disease by using the determined input variables and a training dataset including adverse reaction information for the specific drug and the specific disease.

In one embodiment, building the periodically updated transformed database may include: acquiring the raw data for detecting the adverse drug reaction from electronic medical records (EMR), wherein the raw data including at least one of medication intake related data, disease diagnosis data, diagnostic test data, or patient nursing record data; converting terminology of the raw data to correspond to a common data standard specification of the prediction model; and building the periodically updated transformed database scheduled to be updated at the predetermined time intervals using at least one of a database linkage technique, a file linkage technique, or a change-history distinction linkage technique.

In one embodiment, the raw data may be additionally acquired from at least one of FAERS, KAERS, WHO-ART, SIDER, or EU-ADR.

In one embodiment, the input variables may include at least one of demographic variables, medication intake related variables, diagnostic test related variables, nursing record related variables, or adverse drug reaction diagnosis related variables.

In one embodiment, the demographic variables may include at least one of gender, age, or region; the medication intake related variables may include at least one of drug type, medication period, medication dosage, or medication history; the diagnostic test related variables may include at least one of imaging tests, blood tests, or urine tests; the nursing record related variables may be variables obtained from nursing records using at least one technique of optical character recognition (OCR), natural language processing (NLP), or text mining; and the adverse drug reaction diagnosis related variables may include information related to a disease diagnosis caused by an adverse drug reaction.

In one embodiment, the input variables may be determined using at least one of: a first technique that determines variable importance by using perturbed input data in which at least a portion of the variables are changed and output data output from the prediction model in response to the perturbed input data; a second technique that selects input variables corresponding to principal components by transforming data in a high-dimensional space into a low-dimensional space; or a third technique that selects input variables in a direction that reduces impurity on a decision tree.

In one embodiment, the input variables may be preprocessed based on performing outlier processing, performing missing value imputation, performing categorization or re-categorization, and performing normalization or standardization.

In one embodiment, training the prediction model for detecting the adverse drug reaction may include: determining the prediction model for detecting the adverse drug reaction among a plurality of candidate models; and training the prediction model such that, using the preprocessed input variables as input data, the prediction model outputs whether an adverse drug reaction occurs for a specific drug or a specific disease, or a probability of occurrence of the adverse drug reaction for the specific drug or the specific disease.

In one embodiment, the prediction model may be a model selected from among candidate models including Random Forest, Logistic Regression, Support Vector Machine, Decision Tree, Naive Bayes, k-Nearest Neighbor, XGBoost, LightGBM, CatBoost, Convolutional Neural Network (CNN), Recurrent Neural Network (RNN), Long Short-Term Memory (LSTM), Transformer, and Generative Pre-trained Transformer (GPT).

In one embodiment, the prediction model may be determined based on: obtaining output results from each of the candidate models using a predetermined test dataset; performing performance evaluation on the output results from each of the candidate models; and determining one model among the candidate models as the prediction model based on a result of the performance evaluation.

In one embodiment, the adverse reaction information for the specific drug and the specific disease may include: first information indicating whether an adverse drug reaction occurs for a specific drug; second information indicating a probability of occurrence of an adverse drug reaction for the specific drug; third information indicating whether an adverse drug reaction occurs for a specific disease; and fourth information indicating a probability of occurrence of an adverse drug reaction for the specific disease.

In one embodiment, the method may further include transmitting an output result of the prediction model for the adverse drug reaction to a user terminal.

In one embodiment, the output result transmitted to the user terminal may include at least one of: a first result indicating a binary classification of an adverse drug reaction as detected or not detected; a second result indicating a probability notation for the adverse drug reaction; or a third result indicating a three-level categorical notation linking a drug, an adverse reaction, and a risk level.

In one embodiment, an output result output from the prediction model may be used to retrain the prediction model based on an accuracy evaluation performed on the output result.

In one embodiment, feedback information corresponding to the output result transmitted from a user terminal that has received the output result of the prediction model may be used to retrain the prediction model.

In one embodiment, a computer program stored in a computer-readable medium is disclosed. The computer program, when executed by a computing device, may cause the computing device to perform operations for generating a prediction result for an adverse drug reaction using artificial intelligence technology, the operations comprising: building a periodically updated transformed database scheduled to be updated at predetermined time intervals using raw data for detecting an adverse drug reaction; determining a plurality of input variables for training an artificial intelligence-based prediction model for detecting the adverse drug reaction based on data stored in the periodically updated transformed database; and training the prediction model such that, in response to receiving the determined input variables, the prediction model outputs adverse reaction information for a specific drug and a specific disease by using the determined input variables and a training dataset including adverse reaction information for the specific drug and the specific disease.

A computing device according to one embodiment is disclosed. The computing device may include at least one processor and a memory. The at least one processor may be configured to: build a periodically updated transformed database scheduled to be updated at predetermined time intervals using raw data for detecting an adverse drug reaction; determine a plurality of input variables for training an artificial intelligence-based prediction model for detecting the adverse drug reaction based on data stored in the periodically updated transformed database; and train the prediction model such that, in response to receiving the determined input variables, the prediction model outputs adverse reaction information for a specific drug and a specific disease by using the determined input variables and a training dataset including adverse reaction information for the specific drug and the specific disease.

According to one embodiment, a method for generating a prediction result for an adverse drug reaction using artificial intelligence technology performed by a computing device is disclosed. The method may include: acquiring input data for detecting an adverse drug reaction based on data stored in a periodically updated transformed database scheduled to be updated at predetermined time intervals using raw data for detecting an adverse drug reaction; and acquiring adverse reaction information for a specific drug and a specific disease from the input data using an artificial intelligence-based prediction model.

### Advantageous Effects

According to embodiments of the present disclosure, the method and the device can contribute to managing drug adverse reactions and creating a virtuous cycle structure in the field of drug safety for enabling universal application without distinction between inpatient/outpatient settings or specific drugs.

### DESCRIPTION OF DRAWINGS

FIG. 1 schematically illustrates a block diagram of a computing device according to one embodiment of the present disclosure.

FIG. 2 illustrates an exemplary structure of an artificial intelligence-based model according to one embodiment of the present disclosure.

FIG. 3 exemplarily illustrates a method for generating an output result of a model including a prediction of an adverse drug reaction according to one embodiment of the present disclosure.

FIG. 4 illustrates a flowchart for building a periodic conversion database including principles for periodic data loading and change history table loading of an electronic medical record and a periodic conversion database according to one embodiment of the present disclosure.

FIG. 5 illustrates a concept of file linkage of building a periodic conversion database according to one embodiment of the present disclosure.

FIG. 6 exemplarily illustrates an overall process including database building, development of artificial intelligence for an adverse drug reaction detection, and notification of an abnormality detection result in sequence according to one embodiment of the present disclosure.

FIG. 7 is a schematic view of a computing environment according to one embodiment of the present disclosure.

### BEST MODE

Various embodiments will be described with reference to the drawings. In this specification, various descriptions are provided to aid in understanding the present disclosure. Before explaining specific details for implementing the embodiments of the present disclosure, it should be noted that configurations not directly related to the technical gist of the present disclosure have been omitted within a range that does not detract from the substance of the invention. Furthermore, the terms and words used in this specification and the claims should be interpreted as having meanings and concepts consistent with the technical spirit of the invention, based on the principle that the inventor can appropriately define the concept of terms to best explain the invention.

As used herein, the terms “component,” “module,” “system,” “unit,” and the like refer to a computer-related entity, such as hardware, firmware, software, a combination of software and hardware, or execution of software, and may be used interchangeably. For example, a component may be a process (procedure) executed on a processor, a processor itself, an object, an execution thread, a program, and/or a computer, but is not limited thereto. For example, both an application executed on a computing device and the computing device itself may constitute components. One or more components may reside within a processor and/or a thread of execution. A component may be localized in a single computer, or distributed across two or more computers. Further, such components may be executed by various computer-readable media having various data structures stored therein. The components may communicate through local and/or remote processing based on signals having one or more data packets (for example, data transmitted from one component interacting with another component in a local or distributed system, or data transmitted through a network such as the Internet).

In addition, the term “or” is intended to mean an inclusive “or” rather than an exclusive “or. ” That is, unless otherwise specified or clearly indicated by the context, the phrase “X uses A or B” is intended to mean any of the natural inclusive alternatives—namely, the case where X uses A, the case where X uses B, or the case where X uses both A and B. Furthermore, the term “and/or,” as used herein, should be understood to designate and include all possible combinations of one or more of the listed related items.

In addition, the term “comprise” and/or “including” should be interpreted as indicating the presence of stated features and/or components. However, it should be understood that the terms “comprise” and/or “including” do not exclude the presence or addition of one or more other features, components, and/or groups thereof. Also, unless otherwise specified, or clearly indicated by the context to refer to a singular form, the singular form as used in this specification and the claims should generally be interpreted to mean “one or more.”

Furthermore, the phrase “at least one of A or B” or “at least one of A and B” should be interpreted to mean “a case including only A,” “a case including only B,” and “a case including both A and B in combination.”

Those skilled in the art will further recognize that various exemplary logical components, blocks, modules, circuits, means, logics, and algorithms described in connection with the embodiments disclosed herein may be implemented in electronic hardware, computer software, or a combination of both. To clearly illustrate the interchangeability between hardware and software, various exemplary components, blocks, means, logics, modules, circuits, and steps have been generally described above in terms of their functionality. Whether such functionality is implemented as hardware or software depends on the specific application and design constraints imposed on the overall system. Skilled artisans may implement the described functionality in various ways for each particular application; however, such implementation choices should not be interpreted as departing from the scope of the present disclosure.

The description of the presented embodiments is provided so that those skilled in the art may utilize or implement the present disclosure. Various modifications to these embodiments will be apparent to those skilled in the art. The general principles defined herein may be applied to other embodiments without departing from the scope of the present disclosure. Therefore, the present invention is not limited to the embodiments set forth herein, but should be construed in the broadest scope consistent with the principles and novel features disclosed herein.

In the present disclosure, terms expressed as “first,” “second,” or “third,” or the like (i.e., N-th) are used merely for distinguishing at least one entity. For example, entities designated as first and second may be the same or different from each other.

The term “adverse drug reaction (ADR)” as used in the present disclosure refers to a harmful and unintended reaction that occurs when a pharmaceutical product or the like is administered or used, where a causal relationship with the pharmaceutical product or the like cannot be excluded, and may include not only adverse reactions occurring at normal doses but also adverse drug reactions and withdrawal symptoms that occur when a drug is intentionally or accidentally used in overdose or when a drug is abused, as well as cases where the expected pharmacological action does not appear.

“Drug adverse reaction monitoring” refers to activities for rapidly and systematically collecting or evaluating various adverse events occurring during pharmaceutical use to implement countermeasures and communicate safety information and action results to medical professionals, consumers, and others, thereby establishing rational pharmaceutical use and preventing drug-related harm in advance.

Examples of the ADR may include a side effect and an adverse drug event (ADE). The adverse action is a concept opposite to a principal action that appears when a pharmaceutical is used for a specific purpose, referring to all unintended effects that occur when a drug is administered according to a normal dosage. Since the adverse action means all actions other than those intended for therapeutic purposes, the adverse action is a concept that can be actively utilized in various fields that comprehensively study all actions regardless of the presence or absence of harmfulness.

The adverse drug example (ADE) refers to an undesirable and unintended sign, symptom, or disease that occurs during administration or use of a pharmaceutical product or the like, regardless of a causal relationship with the pharmaceutical product.

The adverse drug reaction (ADR) is a harmful and unintended reaction occurring during administration or use of pharmaceuticals according to the normal dosage, where a causal relationship with the pharmaceutical cannot be excluded, and since the ADR is predictable and preventable in advance, management by pharmacists and medical personnel is considered important.

Drug metabolism occurs mostly in the liver, and a metabolic action takes place in the endoplasmic reticulum of hepatocytes through oxidation, reduction, hydrolysis, hydration, conjugation, condensation, or isomerization reactions of drugs by the cytochrome P450 (CYP450) metabolic enzyme system in endoplasmic reticulum of a liver cell. Four general major parameters of pharmacokinetics include absorption, distribution, metabolism, and excretion. Dose, frequency, route of administration, tissue distribution, and protein binding of drugs to receptors may affect drug metabolism. In addition, pathological factors including gastrointestinal diseases, cardiopulmonary circulatory diseases, respiratory diseases, and renal excretory diseases may also affect the drug metabolism.

Embodiments in the present disclosure may include a step of deriving predictable ADRs in order according to causality (e.g., certain, probable, possible, unlikely, conditional/unassessable) based on the type of drug administered for an indication and demographic background, race/individual genotype/phenotype, medical history, etc., classification according to degree of reaction (e.g., mild, moderate, severe), or frequency of occurrence.

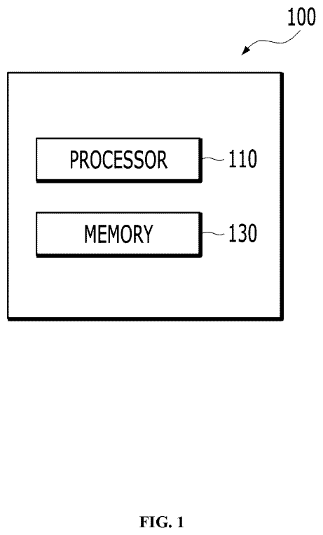

FIG. 1 schematically illustrates a block diagram of a computing device 100 according to one embodiment of the present disclosure.

The computing device 100 according to one embodiment of the present disclosure may include a processor 110 and a memory 130.

A configuration of the computing device 100 illustrated in FIG. 1 is only a simplified example. In one embodiment of the present disclosure, the computing device 100 may include other components for performing the computing environment of the computing device 100, and only some of the disclosed components may constitute the computing device 100.

In the present disclosure, the computing device 100 may mean any type of node constituting a system for implementing embodiments of the present disclosure. The computing device 100 may mean any type of user terminal or any type of server. The components of the computing device 100 may be exemplary and some components may be excluded, or an additional component may be included in the computing device 100. As an example, when the computing device 100 includes a user terminal, an output unit (not illustrated) and an input unit (not illustrated) may be included in a scope of the computing device 100.

The computing device 100 in the present disclosure may perform technical features according to embodiments of the present disclosure to be described below. For example, the computing device 100 may generate prediction results including adverse drug reactions (ADRs) that may occur in a patient corresponding to the input data (e.g., a specific drug triggers excessive immune system activity in a specific individual, causing a cellular immune response cascade) using an artificial intelligence-based prediction model that utilizes input data corresponding to the patient's medical history, drug allergy history, administered drugs, and dosages.

As an additional example, the computing device 100 may obtain information about the patient's demographic information, drug usage history, medical history, and blood test data (e.g., 65 years old, Asian male, administration of serotonin reuptake inhibitors (SSRIs) and divalproate for the past 26 months, and statins-based drug for the past 7 months, and abdominal pain and constipation experienced as drug adverse events) from the database, and based on the obtained information, generate ADR prediction results corresponding to the drug or specific disease to provide corresponding materials including cost-benefit analysis of drug use.

As an additional example, the computing device 100 may more efficiently collect raw data from multiple medical institutions by building a periodic conversion database that is converted to common data model specifications using electronic medical records. The computing device 100 may be configured to perform detection of more recent adverse drug reactions by utilizing a periodically updated periodic conversion database.

In one embodiment of the present disclosure, the computing device 100 may obtain a result of nucleotide sequence analysis (e.g., Next Generation Sequencing) from a server or external entity. In another embodiment, the computing device 100 may perform the nucleotide sequence analysis on proteomes and genes (e.g., DNA or RNA) obtained from biological samples derived from a subject. As used in the present disclosure, the term “nucleotide sequence analysis” may be performed by any form of technique capable of analyzing nucleotide sequences, including, for example, whole genome sequencing, whole exome sequencing, or whole transcriptome sequencing, but is not limited thereto.

The term “subject” as used in the present disclosure may refer to an object or individual for obtaining a biological sample including saliva, hair follicles, proteomes, genes and/or combinations thereof.

As used in the present disclosure, the term “sample” may be used without limitation as long as the sample is obtained from a subject or individual for which a gene genome and/or gene phenotype is to be determined, and may be, for example, cells or tissues obtained by biopsy, blood, whole blood, serum, plasma, saliva, cerebrospinal fluid, various secretions, urine and/or feces. Preferably, the sample may be selected from the group consisting of blood, plasma, serum, saliva, nasal fluid, sputum, ascites, vaginal secretions and/or urine, and more preferably may be blood, plasma or serum. The sample may be pretreated before use for detection or diagnosis. For example, the pretreatment method may include homogenization, filtration, distillation, extraction, concentration, inactivation of interfering components, and/or addition of reagents. In the present disclosure, the biological sample may be, but is not limited to, tissue, cells, whole blood, and/or blood.

In one embodiment, the processor 110 may be constituted by at least one core, and may include processors for data analysis and/or processing, which include a central processing unit (CPU), a general purpose graphics processing unit (GPGPU), a tensor processing unit (TPU), and the like of the computing device 100.

The processor 110 may read a computer program stored in the memory 130 to generate prediction results including occurrence and/or probability of adverse drug events (ADEs) according to one embodiment of the present disclosure.

According to one embodiment of the present disclosure, the processor 110 may also perform a computation for learning a neural network. The processor 110 may perform calculations for learning the neural network, which include processing of input data for learning in deep learning (DL), extracting a feature in the input data, calculating an error, updating a weight of the neural network using backpropagation, and the like. At least one of the CPU, GPGPU, and TPU of the processor 110 may process learning of a network function. For example, both the CPU and the GPGPU may process the learning of the network function and data classification using the network function. Further, in one embodiment of the present disclosure, processors of the plurality of computing devices may be used together to process the learning of the network function and the data classification using the network function. Further, the computer program executed in the computing device according to one embodiment of the present disclosure may be a CPU, GPGPU, or TPU executable program.

Additionally, the processor 110 may generally process an overall operation of the computing device 100. For example, the processor 110 may process data, information, signals, or the like input or output through the components included in the computing device 100 or drive the application program stored in a storage unit to provide the user with or process appropriate information or function.

According to one embodiment of the present disclosure, the memory 130 may store any type of information generated or determined by the processor 110 or any type of information received by the computing device 100. According to one embodiment of the present disclosure, the memory 130 may be a storage medium that stores computer software which allows the processor 110 to perform the operations according to the embodiments of the present disclosure. Therefore, the memory 130 may mean computer-readable media for storing software codes required for performing the embodiments of the present disclosure, data which become execution targets of the codes, and execution results of the codes.

According to one embodiment of the present disclosure, the memory 130 may mean any type of storage medium, and examples thereof include at least one type of storage medium of a flash memory type storage medium, a hard disk type storage medium, a multimedia card micro type storage medium, a card type memory (for example, an SD or an XD memory, or the like), a random access memory (RAM), a static random access memory (SRAM), a read-only memory (ROM), an electrically erasable programmable read-only memory (EEPROM), a programmable read-only memory (PROM), a magnetic memory, a magnetic disk, and an optical disk. The computing device 100 may operate in connection with a web storage performing a storing function of the memory 130 on the Internet. The foregoing description of memory is merely an example, and the memory 130 used in the present disclosure is not limited to the aforementioned examples.

In the present disclosure, the communication unit (not illustrated) may be configured regardless of communication modes such as wired and wireless modes and constituted by various communication networks including a personal area network (PAN), a wide area network (WAN), and the like. Further, the communication unit may operate based on known World Wide Web (WWW) and may adopt a wireless transmission technology used for short-distance communication, such as infrared data association (IrDA) or Bluetooth.

The computing device 100 in the present disclosure may include any type of user terminal and/or any type of server. Therefore, the embodiments of the present disclosure may be performed by the server and/or the user terminal.

The user terminal may include any type of terminal which is capable of interacting with the server or another computing device. Examples of the user terminal may include a mobile phone, a smart phone, a laptop computer, personal digital assistants (PDA), a slate PC, a tablet PC, and an ultra-book. The server may include any type of computing system or computing device, such as a microprocessor, a mainframe computer, a digital processor, a portable device, and a device controller.

In an additional embodiment, the aforementioned server may refer to an entity that stores and manages drug administration information and reactions thereto, electronic medical records (EMR), diagnostic test data, disease diagnosis data, nursing records, and the like. The server may more efficiently collect medical data from multiple medical institutions through a database converted to common data model specifications, and include a storage unit (not shown) for storing relevant information to enable detection of the latest adverse drug reactions by utilizing a regularly updated database. As an example, the storage unit may be included in the server, or may be present under the management of the server. As another example, the storage unit may also be present outside the server, and implemented in a form which is capable of communicating with the server. In this case, the storage unit may be managed and controlled by another external server different from the server.

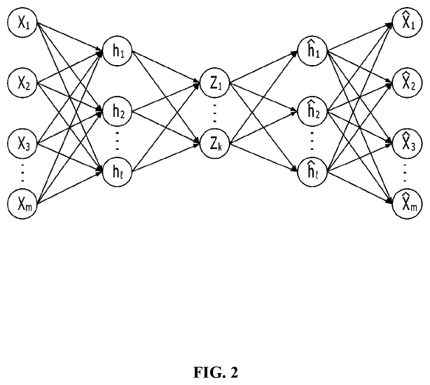

FIG. 2 illustrates a network function according to one embodiment of the present disclosure.

Throughout the present specification, a prediction model, an artificial intelligence-based prediction model, an artificial intelligence model, an artificial intelligence-based model, an operation model, a neural network, a network function, and a neural network may be used interchangeably to refer to the same concept.

The neural network may be generally constituted by an aggregate of calculation units which are mutually connected to each other, which may be called “node”. The nodes may also be called neurons. The neural network is configured to include one or more nodes. The nodes (or neurons) constituting the neural networks may be mutually connected to each other by one or more links.

In the neural network, one or more nodes connected through the link may relatively form a relationship between an input node and an output node. Concepts of the input node and the output node are relative and a predetermined node which has the relationship of the output node with respect to one node may have the relationship of the input node in the relationship with another node and vice versa. As described above, the relationship of the output node to the input node may be generated based on the link. One or more output nodes may be connected to one input node through the link and vice versa.

In the relationship of the input node and the output node connected through one link, a value of data of the output node may be determined based on data input in the input node. Here, a link connecting the input node and the output node to each other may have a weight. The weight may be variable, and the weight may be varied by a user or an algorithm in order for the neural network to perform a desired function. For example, when one or more input nodes are mutually connected to one output node by the respective links, the output node may determine an output node value based on values input in the input nodes connected with the output node and the weights set in the links corresponding to the respective input nodes.

As described above, in the neural network, one or more nodes are connected to each other through one or more links to form the input node and output node relationship in the neural network. A characteristic of the neural network may be determined according to the number of nodes, the number of links, correlations between the nodes and the links, and values of the weights granted to the respective links. For example, when the same number of nodes and links exist and two neural networks in which the weight values of the links are different from each other exist, it may be recognized that two neural networks are different from each other.

The neural network may be constituted by a set of one or more nodes. A subset of the nodes constituting the neural network may constitute a layer. Some of the nodes constituting the neural network may constitute one layer based on the distances from the initial input node. For example, a set of nodes of which distance from the initial input node is n may constitute n layers. The distance from the initial input node may be defined by the minimum number of links which should be passed from the initial input node up to the corresponding node. However, definition of the layer is predetermined for description and the order of the layer in the neural network may be defined by a method different from the aforementioned method. For example, the layers of the nodes may be defined by the distance from a final output node.

In one embodiment of the present disclosure, the set of the neurons or the nodes may be defined as the expression “layer”.

The initial input node may mean one or more nodes in which data is directly input without passing through the links in the relationships with other nodes among the nodes in the neural network. Alternatively, in the neural network, in the relationship between the nodes based on the link, the initial input node may mean nodes which do not have other input nodes connected through the links. Similarly thereto, the final output node may mean one or more nodes which do not have the output node in the relationship with other nodes among the nodes in the neural network. Further, a hidden node may mean not the initial input node and the final output node but the nodes constituting the neural network.

In the neural network according to one embodiment of the present disclosure, the number of nodes of the input layer may be the same as the number of nodes of the output layer, and the neural network may be a neural network of a type in which the number of nodes decreases and then, increases again from the input layer to the hidden layer. Further, in the neural network according to another exemplary embodiment of the present disclosure, the number of nodes of the input layer may be smaller than the number of nodes of the output layer, and the neural network may be a neural network of a type in which the number of nodes increases from the input layer to the hidden layer. Further, in the neural network according to yet another exemplary embodiment of the present disclosure, the number of nodes of the input layer may be larger than the number of nodes of the output layer, and the neural network may be a neural network of a type in which the number of nodes decreases from the input layer to the hidden layer. The neural network according to still yet another exemplary embodiment of the present disclosure may be a neural network of a type in which the neural networks are combined.

The deep neural network (DNN) may mean a neural network including a plurality of hidden layers other than the input layer and the output layer. When the deep neural network is used, the latent structures of data may be identified. The deep neural network may include convolutional neural network (CNN), recurrent neural network (RNN), auto encoder, generative adversarial networks (GAN), restricted Boltzmann machine (RBM), deep belief network (DBN), Q network, U network, Siamese network, etc. The description of the deep neural network described above is just an example and the present disclosure is not limited thereto.

Exemplarily, the artificial intelligence-based prediction model of the present disclosure may include a Random Forest, Logistic Regression, Support Vector Machine, Decision Tree, Naive Bayes, k-Nearest Neighbor, XGBoost, LightGBM, CatBoost, Recurrent Neural Network (RNN), Long Short-Term Memory (LSTM) network, Bidirectional Long Short-Term Memory (BiLSTM) network, Transformer, Generative Pre-trained Transformer (GPT), BERT (Bidirectional Encoder Representations from Transformers), SpanBERT, Gated Recurrent Unit (GRU), or Bidirectional Gated Recurrent Unit (BiGRU).

The artificial intelligence based model of the present disclosure may be expressed by a network structure of an arbitrary structure described above, including the input layer, the hidden layer, and the output layer.

The neural network which may be used in the artificial intelligence based model of the present disclosure may be learned in at least one scheme of supervised learning, unsupervised learning, semi supervised learning, or reinforcement learning, federated learning for distributed deep learning, and incremental learning. The learning of the neural network may be a process in which the neural network applies knowledge for performing a specific operation to the neural network. For example, a prediction model according to one embodiment of the present disclosure may be trained using a semi-supervised learning method in which a mask is applied to a patient's genomic information, administered drugs, and symptoms indicative of potential adverse drug reactions, or at least a portion of such symptoms, and the model is trained to predict the masked symptoms (e.g., adverse drug reactions). In this case, at least a portion of the administered drugs and potential adverse drug reactions may be used as labeled data for training the prediction model, while the remaining portion may be used as unlabeled data for training the prediction model.

The neural network may be learned in a direction to minimize errors of an output. The learning of the neural network is a process of repeatedly inputting learning data into the neural network and calculating the output of the neural network for the learning data and the error of a target and back-propagating the errors of the neural network from the output layer of the neural network toward the input layer in a direction to reduce the errors to update the weight of each node of the neural network. In the case of the supervised learning, the learning data labeled with a correct answer is used for each learning data (i.e., the labeled learning data) and in the case of the unsupervised learning, the correct answer may not be labeled in each learning data. That is, for example, the learning data in the case of the supervised learning related to the data classification may be data in which category is labeled in each learning data. The labeled learning data is input to the neural network, and the error may be calculated by comparing the output (category) of the neural network with the label of the learning data.

As another example, in the case of the unsupervised learning related to the data classification, the learning data as the input is compared with the output of the neural network to calculate the error. The calculated error is back-propagated in a reverse direction (i.e., a direction from the output layer toward the input layer) in the neural network and connection weights of respective nodes of each layer of the neural network may be updated according to the back propagation. A variation amount of the updated connection weight of each node may be determined according to a learning rate. Calculation of the neural network for the input data and the back-propagation of the error may constitute a learning cycle (epoch). The learning rate may be applied differently according to the number of repetition times of the learning cycle of the neural network. For example, in an initial stage of the learning of the neural network, the neural network ensures a certain level of performance quickly by using a high learning rate, thereby increasing efficiency and uses a low learning rate in a latter stage of the learning, thereby increasing accuracy.

In learning of the neural network, the learning data may be generally a subset of actual data (i.e., data to be processed using the learned neural network), and as a result, there may be a learning cycle in which errors for the learning data decrease, but the errors for the actual data increase. Overfitting is a phenomenon in which the errors for the actual data increase due to excessive learning of the learning data. For example, a phenomenon in which the neural network that learns a cat by showing a yellow cat sees a cat other than the yellow cat and does not recognize the corresponding cat as the cat may be a kind of overfitting. The overfitting may act as a cause which increases the error of the machine learning algorithm. Various optimization methods may be used in order to prevent the overfitting. In order to prevent the overfitting, a method such as increasing the learning data, regularization, dropout of omitting a part of the node of the network in the process of learning, utilization of a batch normalization layer, etc., may be applied.

According to one embodiment of the present disclosure, a computer-readable medium storing a data structure is disclosed. The above-described data structure may be stored in the memory of the present disclosure, executed by the processor, and transmitted or received by the network unit.

The data structure may refer to the organization, management, and storage of data that enables efficient access to and modification of data. The data structure may refer to the organization of data for solving a specific problem (e.g., data search, data storage, data modification in the shortest time). The data structures may be defined as physical or logical relationships between data elements, designed to support specific data processing functions. The logical relationship between data elements may include a connection relationship between data elements that the user defines. The physical relationship between data elements may include an actual relationship between data elements physically stored on a computer-readable storage medium (e.g., persistent storage device). The data structure may specifically include a set of data, a relationship between the data, a function which may be applied to the data, or instructions. Through an effectively designed data structure, a computing device may perform operations while using the resources of the computing device to a minimum. Specifically, the computing device may increase the efficiency of operation, read, insert, delete, compare, exchange, and search through the effectively designed data structure.

The data structure may be divided into a linear data structure and a non-linear data structure according to the type of data structure. The linear data structure may be a structure in which only one data is connected after one data. The linear data structure may include a list, a stack, a queue, and a deque. The list may mean a series of data sets in which an order exists internally. The list may include a linked list. The linked list may be a data structure in which data is connected in a scheme in which each data is linked in a row with a pointer. In the linked list, the pointer may include link information with next or previous data. The linked list may be represented as a single linked list, a double linked list, or a circular linked list depending on the type. The stack may be a data listing structure with limited access to data. The stack may be a linear data structure that may process (e.g., insert or delete) data at only one end of the data structure. The data stored in the stack may be a data structure (LIFO-Last in First Out) in which the data is input last and output first. The queue is a data listing structure that may access data limitedly and unlike a stack, the queue may be a data structure (FIFO-First in First Out) in which late stored data is output late. The deque may be a data structure capable of processing data at both ends of the data structure.

The non-linear data structure may be a structure in which a plurality of data are connected after one data. The non-linear data structure may include a graph data structure. The graph data structure may be defined as a vertex and an edge, and the edge may include a line connecting two different vertices. The graph data structure may include a tree data structure. The tree data structure may be a data structure in which there is one path connecting two different vertices among a plurality of vertices included in the tree. That is, the tree data structure may be a data structure that does not form a loop in the graph data structure.

Throughout this specification, a prediction model, an artificial intelligence-based model, an operation model, a neural network, a network function, and a neural network may be used interchangeably. Hereinafter, the term “neural network” will be used consistently. The data structure may include the neural network. In addition, the data structures, including the neural network, may be stored in a computer readable medium. The data structure including the neural network may also include data preprocessed for processing by the neural network, data input to the neural network, weights of the neural network, hyper parameters of the neural network, data obtained from the neural network, an active function associated with each node or layer of the neural network, and a loss function for learning the neural network. The data structure including the neural network may include predetermined components of the components disclosed above. In other words, the data structure including the neural network may include all of data preprocessed for processing by the neural network, data input to the neural network, weights of the neural network, hyper parameters of the neural network, data obtained from the neural network, an active function associated with each node or layer of the neural network, and a loss function for learning the neural network or a combination thereof. In addition to the above-described configurations, the data structure including the neural network may include predetermined other information that determines the characteristics of the neural network. In addition, the data structure may include all types of data used or generated in the calculation process of the neural network, and is not limited to the above. The computer readable medium may include a computer readable recording medium and/or a computer readable transmission medium. The neural network may be generally constituted by an aggregate of calculation units which are mutually connected to each other, which may be called “node”. The nodes may also be called neurons. The neural network is configured to include one or more nodes.

The data structure may include data input into the neural network. The data structure including the data input into the neural network may be stored in the computer readable medium. The data input to the neural network may include learning data input in a neural network learning process and/or input data input to a neural network in which learning is completed. The data input to the neural network may include preprocessed data and/or data to be preprocessed. The preprocessing may include a data processing process for inputting data into the neural network. Therefore, the data structure may include data to be preprocessed and data generated by preprocessing. The data structure is just an example and the present disclosure is not limited thereto.

The data structure may include the weight of the neural network (in the present disclosure, the weight and the parameter may be used as the same meaning). In addition, the data structures, including the weight of the neural network, may be stored in the computer readable medium. The neural network may include a plurality of weights. The weight may be variable and the weight may be varied by a user or an algorithm in order for the neural network to perform a desired function. For example, when one or more input nodes are mutually connected to one output node by the respective links, the output node may determine a data value output from an output node based on values input in the input nodes connected with the output node and the weights set in the links corresponding to the respective input nodes. The data structure is just an example and the present disclosure is not limited thereto.

As a non-limiting example, the weight may include a weight which varies in the neural network learning process and/or a weight in which neural network learning is completed. The weight which varies in the neural network learning process may include a weight at a time when a learning cycle starts and/or a weight that varies during the learning cycle. The weight in which the neural network learning is completed may include a weight in which the learning cycle is completed. Accordingly, the data structure including the weight of the neural network may include a data structure including the weight which varies in the neural network learning process and/or the weight in which neural network learning is completed. Accordingly, the above-described weight and/or a combination of each weight are included in a data structure including a weight of a neural network. The data structure is just an example and the present disclosure is not limited thereto.

The data structure including the weight of the neural network may be stored in the computer-readable storage medium (e.g., memory, hard disk) after a serialization process. Serialization may be a process of storing data structures on the same or different computing devices and later reconfiguring the data structure and converting the data structure to a form that may be used. The computing device may serialize the data structure to send and receive data over the network. The data structure including the weight of the serialized neural network may be reconfigured in the same computing device or another computing device through deserialization. The data structure including the weight of the neural network is not limited to the serialization. Furthermore, the data structure including the weight of the neural network may include a data structure (for example, B-Tree, R-Tree, Trie, m-way search tree, AVL tree, and Red-Black Tree in a nonlinear data structure) to increase the efficiency of operation while using resources of the computing device to a minimum. The above-described matter is just an example and the present disclosure is not limited thereto.

The data structure may include hyper-parameters of the neural network. In addition, the data structures, including the hyper-parameters of the neural network, may be stored in the computer readable medium. The hyper-parameter may be a variable which may be varied by the user. The hyper-parameter may include, for example, a learning rate, a cost function, the number of learning cycle iterations, weight initialization (for example, setting a range of weight values to be subjected to weight initialization), and Hidden Unit number (e.g., the number of hidden layers and the number of nodes in the hidden layer). The data structure is just an example, and the present disclosure is not limited thereto.

As a network function for the prediction model according to one embodiment of the present disclosure, a transformer may be considered. For example, the prediction model may operate based on a transformer. Such a prediction model may, for instance, operate using a recurrent neural network with an attention algorithm applied, or a transformer with an attention algorithm applied.

In one embodiment, the transformer may be constituted by an encoder that encodes embedded data and a decoder that decodes the encoded data. The transformer may have a structure that receives a series of data and outputs a series of data of a different type through encoding and decoding steps. In one embodiment, the series of data may be processed into a form operable by the transformer. A process of processing the series of data into a form operable by the transformer may include an embedding process. Expressions such as data token, embedding vector, or embedding token may refer to the embedded data in a form that can be processed by the transformer.

To encode and decode a series of data, the transformer may process the encoder and decoder within the transformer by utilizing an attention algorithm. The attention algorithm may refer to an algorithm that, for a given query, calculates similarities to one or more keys, applies the calculated similarities to the values corresponding to each key, and then computes an attention value by weighting and summing the values with the applied similarities.

Depending on how the query, key, and value are set, various types of attention algorithms may be classified. For example, if the query, key, and value are all set to be identical when computing attention, this may refer to a self-attention algorithm. To process the input series of data in parallel, if the embedding vectors are reduced in dimension and individual attention heads are calculated for each divided embedding vector to compute attention, this may refer to a multi-head attention algorithm.

In one embodiment, the transformer may include modules that perform multiple multi-head self-attention algorithms or multi-head encoder-decoder algorithms. In one embodiment, the transformer may also include additional components that are not attention algorithms, such as an embedding layer, a normalization layer, and a softmax layer. Methods of constructing a transformer using an attention algorithm may include the method disclosed in Vaswani et al., Attention Is All You Need, 2017 NIPS, which is incorporated herein by reference.

The transformer may be applied to various data domains, such as embedded natural language, embedded sequence information, segmented image data, or audio waveforms, to convert a series of input data into a series of output data. To transform data from various domains into a series of data that can be input to the transformer, the transformer may embed the data. The transformer may process additional data representing the relative positional or phase relationships between the series of input data. Alternatively, vectors representing the relative positional or phase relationships between the input data may be additionally incorporated when embedding the series of input data. In one example, the relative positional relationships between the series of input data may include, but are not limited to, word order within a natural language sentence, relative positions of each segmented image, and the temporal order of segmented audio waveforms. A process of adding information representing the relative positional or phase relationships between the series of input data may be referred to as positional encoding.

An example of a method for embedding data and converting it for input to a transformer is disclosed in Dosovitskiy et al., AN IMAGE IS WORTH 16×16 WORDS: TRANSFORMERS FOR IMAGE RECOGNITION AT SCALE, which is incorporated herein by reference.

In the present disclosure, the terms “computing device” and “computing apparatus” may be used interchangeably.

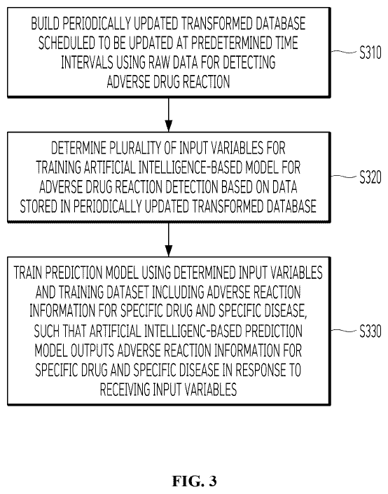

FIG. 3 exemplarily illustrates a method for generating an output result of a model including a prediction of an adverse drug reaction according to one embodiment of the present disclosure.

Embodiments of the present disclosure may encompass embodiments for training a prediction model and embodiments for obtaining inference results (e.g., predictions including possible adverse drug reactions and/or their occurrence probabilities) through the trained prediction model.

In the present disclosure, the operation of the prediction model may be described as one process among either the process of training the prediction model or the process of performing inference on adverse drug reactions using the prediction model (e.g., a process of generating prediction results). This is written for convenience of description, and even if described as one process among either the process of performing training or the process of performing inference, this should be interpreted as intended to encompass the other process among training or inference.

In one embodiment, the steps illustrated in FIG. 3 may be performed by the computing device 100. In an additional embodiment, like a scheme in which some of the steps illustrated in FIG. 3 are performed by the user terminal and some other steps are performed by the server, the steps in FIG. 3 may also be implemented by a plurality of entities.

In one embodiment of the present disclosure, the computing device 100 may build a periodically updated transformed database scheduled to be updated at predetermined time intervals using raw data for detecting an adverse drug reaction (S310).

In one embodiment, the raw data may refer to data obtained before being converted into a database. For example, the raw data may be used to refer to data obtained from various channels such as electronic medical records, etc.

In one embodiment, the raw data may also be obtained from at least one of FAERS, KAERS, WHO-ART, SIDER or EU-ADR.

In one embodiment, the raw data may be obtained from an electronic medical record (EMR). The raw data may include at least one of medication intake related data, disease diagnosis data, diagnostic test data, or nursing record data. For example, the diagnostic test data may refer to data obtained through any form of testing or diagnosis. As an example, the diagnostic test data may include a genetic test record. Specific examples of the diagnostic test data, the medication intake related data, the disease diagnosis data, and the nursing record data will be described below.

In one embodiment, the computing device 100 may receive a predictable adverse drug reaction list (ADR list) from an external rule-based or artificial intelligence-based prediction model that is different from the prediction model according to the present disclosure.

In one embodiment, the provided predictable adverse drug reactions (ADRs) may be sorted based on specific criteria (e.g., causality and/or degree of reaction such as Mild, Moderate, Severe) based on the raw data, and the computing device 100 may additionally process the provided ADRs in addition to the ‘probability of occurrence’ of the ADRs as output data.

In one embodiment, the computing device 100 may convert terms included in the raw data to correspond to common data standard specifications of the database or prediction model. For example, dystonia, athetosis, choreoathetosis, and chorea may be defined as similar or identical ADRs within the same category, and the conversion may be provided with clear distinction from causes other than drug-related causes, which are distinguished from such abnormal movements suspected to be adverse drug reactions. For example, the computing device 100 may classify cerebral palsy, focal intracranial diseases, Huntington's disease, hepatic encephalopathy, hepatic cirrhosis, etc., which are different from the aforementioned athetosis, as diseases or ADRs that are distinguished from dystonia, athetosis, choreoathetosis, and chorea. As an additional example, when the cause of the abnormal movements is not specifically described in the nursing record data, the computing device 100 may not use the data as training data for adverse drug reaction prediction, or may classify and convert the terms as ADRs with low causality with drugs.

In one embodiment according to the present disclosure, the periodic conversion database may refer to a data repository configured to store and update information such as patients' demographic information, drugs and indications they receive, adverse drug events (ADEs) that occur, medical history, etc., at predetermined or optimal time intervals determined by a prediction model. The periodic conversion database may refer to a data repository that collects and builds comprehensive data on newly entered or additionally entered ADR and patient information. For example, a user of the periodic conversion database may search for types of drugs that cause specific ADRs such as seizures-and when searching for bupropion drugs that a patient is taking, may receive statistical summaries of possible other ADRs or dosages of clinical cases that showed seizure symptoms after administration, and patient information. As an example, the artificial intelligence-based model according to one embodiment of the present disclosure may include a model for determining a predetermined time period for conversion or updating in the periodic conversion database. An artificial intelligence-based model that uses, as an input, the amount of data stored in the database, the type of data, and/or the content of the data is operable to output the timing of periodic conversion in the periodic conversion database.

In one embodiment, the computing device 100 may build a periodic conversion database using at least one technique selected from a database linkage technique, a file linkage technique, or a change-history distinction linkage technique.

In one embodiment, the database linkage technique may refer to a data automation method that enables accurate or efficient linkage of disease onset age, duration in months, symptom improvement age, and cure age, for example, based on information about the patient's age at the time of data entry or data acquisition, disease occurrence time, and disease duration.

In one embodiment, the file linkage technique may refer to a method for linking electronic medical record (EMR) documents to a file format most suitable for operating a prediction model and simultaneously operating with an external model different from the prediction model, without loss of information contained therein, even when EMR document extensions differ from each other.

In one embodiment, the change-history distinction linkage technique may refer to a method for organically classifying and linking ADR-related changes according to information such as newly occurring symptoms, disappeared symptoms, symptom severity, added or excluded medications, smoking status, drinking status, and newly discovered medical history for the same patient.

In one embodiment according to the present disclosure, the update cycle of the periodic conversion database that is updated using at least one of the database linkage technique, the file linkage technique, or the change-history distinction linkage technique may be based on a predetermined time period as its unit. As an example, the update cycle of the periodic conversion database may be changed from once a month to once a week according to user needs, or the update cycle may be shortened or extended according to resource sufficiency/insufficiency.

In one embodiment, the computing device 100 may obtain one or more of demographic data, medication intake related data, disease diagnosis data, diagnostic test data, and nursing record data from a public database (e.g., FAERS, KAERS, WHO-ART, SIDER, EU-ADR, etc.). The public database is not limited to the disclosed databases.

In an additional embodiment, the diagnostic test related data may include, for example, gene sequences experimentally sequenced from specimens obtained from patients.

In one embodiment, the computing device 100 may utilize any form of big data analysis technique in building the periodic conversion database. The big data analysis technique may include extracting or generating predictive information by identifying patterns and relationships hidden in large amounts of data. For example, the big data analysis technique may include text mining that extracts specific information existing in unstructured documents, opinion mining that extracts opinions on a specific topic by crawling information related to the specific topic through websites, and/or web mining that extracts desired information from web logs and/or search terms.

In one embodiment of the present disclosure, the computing device 100 may determine a plurality of input variables for training an artificial intelligence-based model for adverse drug reaction detection based on data stored in the periodic conversion database (S320).

In one embodiment, the input variable may refer to a variable or feature used for training or inference of the artificial intelligence model. For example, the input variable may include at least one of a demographic variable, a medication intake related variable, a diagnostic test related variable, a nursing record related variable, or an adverse drug reaction diagnosis related variable.

In one embodiment, the demographic variable is a variable for quantitatively or qualitatively representing population-related phenomena. The demographic variable may include, for example, at least one of age or region.

In one embodiment, the medication intake variable may represent a quantitative or qualitative indicator that may be defined in relation to the user's medication intake. For example, the medication intake related variable may include at least one of drug type, medication period, medication dosage, or medication history.

In one embodiment, the diagnostic test related variable may represent a quantitative or qualitative indicator that may be derived from the results of various forms of diagnosis or examination. For example, the diagnostic test related variable may include at least one of imaging examination, blood test, or urine test.

In one embodiment, the nursing record related variable may mean a quantitative or qualitative variable obtained from nursing records generated during the treatment and/or nursing process. For example, the nursing record related variable may be generated based on information contained in medical or nursing record charts in the hospital. For example, the nursing record-related variable may be obtained through at least one technique of optical character recognition (OCR), natural language processing (NLP), or text mining. In an additional embodiment, the adverse drug reaction diagnosis related variable may include information related to disease diagnosis caused by the drug adverse reactions.

In one embodiment, imaging examination data that may be included in the diagnostic test related variable may be obtained from ultrasound imaging, X-ray (plain radiography), computed tomography (CT), magnetic resonance imaging (MRI), functional magnetic resonance imaging (fMRI), positron emission tomography (PET), and the like.

In one embodiment, information related to the diagnosis of a disease due to the adverse drug reaction, which may be included in variables related to adverse drug reaction diagnosis, may refer to data collected when unintended or unexpected symptoms arising from an administered drug are diagnosed as a disease. In an additional embodiment, the prediction model according to the present disclosure may suggest coadministration to prevent unintended symptoms that may arise from the administered drug.

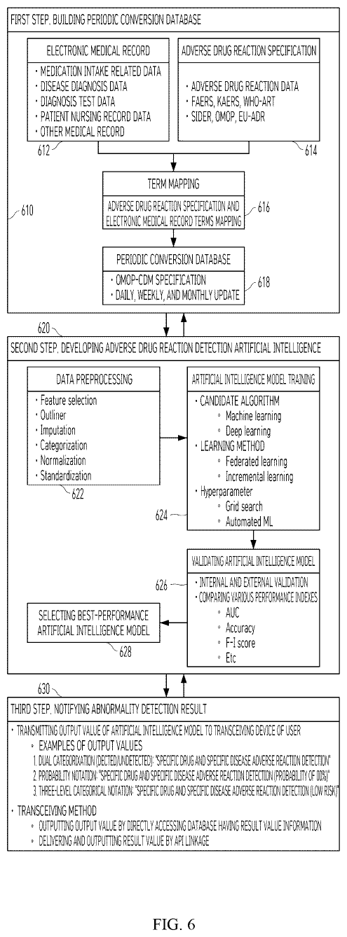

A more detailed description of the input variable selection method will be provided later with reference to FIG. 6.

In one embodiment, parameter information related to a preprocessing method may be obtained from data output from a separate external prediction model that is different from the prediction model of the present disclosure.

In an additional example, preprocessing for training or inference of the prediction model of the present disclosure (e.g., preprocessing process of blood test data variables) may be performed using parameters generated by an external prediction model that operates with patient demographic data and disease information as input data. As an example, when a 36-year-old Caucasian woman is a triple-negative breast cancer (TNBC) patient (HER-2, ER, PR negative), parameter information generated by the external prediction model may increase prediction accuracy for a probability of occurrence of specific ADRs (e.g., mucosal inflammation, erythrodysesthesia, increased blood ALT (Alanine aminotransferase) levels).

In an additional embodiment, the ADR prediction model may be operated by receiving real-time updates of reports from new research papers, as well as databases on administered drugs and specific indications for a particular individual, and adverse drug reactions that have occurred (e.g., FAERS, KAERS, WHO-ART, SIDER, and EU-ADR). In an additional embodiment, the ADR prediction model may be operated by receiving real-time updates of clinical response and progress data entered into international clinical trial databases (e.g., ClinicalTrials.gov), and may also be operated by receiving real-time updates of result reports from research literature on genes and drug responses in human or non-human entities.

In one embodiment, data that serves as an operational basis for the artificial intelligence or rule-based external prediction model that provides parameter information input to the ADR prediction model (e.g., combined processed data such as demographic information, genetic information, administered drugs, and known/unknown adverse effects) is merely an example and is not limited to the present disclosure.

In one embodiment of the present disclosure, the computing device 100 may perform preprocessing to apply a mask to at least one of data corresponding to the adverse drug reaction (ADR), data related to medication duration, and portions of demographic information (e.g., gender, age of onset, and race) for training and/or inference of the prediction model.

In one embodiment, the computing device 100 may determine a category of a region to be masked in the training dataset. For example, the region to be masked may include data of single or multiple categories. The computing device (100) may cause the prediction model to perform masking-based learning based on the determined masking target category. In an additional embodiment, such masking-based preprocessing and training of the prediction model may contribute to, for example, more accurately outputting predicted values of multiple adverse drug reactions (ADRs) in descending order according to an occurrence probability when two patients with similar medical histories differ only in gender, or when the patients have the same gender but different age groups.

In one embodiment, the computing device 100 may determine input variables for the prediction model from among multiple variables based on a first technique that determines variable importance by using perturbated input data in which at least some variables are modified and output data generated from the prediction model in response to the perturbated input data. For example, the computing device 100 may generate perturbated input data acquired by modifying at least some of the multiple variables included in the input data. The computing device 100 may input multiple input data including unmodified input data and perturbated input data with modified variables into an artificial intelligence-based model, and determine which variables among the multiple variables will be selected by comparing the model outputs. For example, the computing device (100) may determine the importance of the modified variable by comparing the model outputs (first output and second output) of first input data with an unmodified first variable and first perturbated input data with a modified first variable. Based on this importance, input variables to be used in the artificial intelligence-based prediction model may be determined. In one embodiment, the computing device 100 may determine input variables by inputting multiple input data including input data with unmodified variables and perturbated input data with modified variables into each of multiple artificial intelligence-based models.

In one embodiment, the computing device 100 may determine input variables for the prediction model from among a plurality of variables based on a second technique that selects input variables corresponding to principal components by transforming data from a high-dimensional space to a low-dimensional space.

In one embodiment, the computing device 100 may determine input variables for the prediction model among a plurality of variables based on a third technique that selects input variables in a direction that reduces impurity on a decision tree.

In one embodiment, the computing device 100 may determine input variables based on Boruta SHAP, Principal Component Analysis (PCA), Feature importance, Permutation importance, and/or Gini importance. Such determination of input variables may be included in the preprocessing process.

In one embodiment, the preprocessing process may be performed based additionally on at least one of: a technique for processing outliers, a technique for performing imputation, a technique for performing categorization or re-categorization, and a technique for performing normalization or standardization. Through these processes, input variables may be determined and/or preprocessed. The preprocessed input variables may be input to the prediction model to perform training and/or inference.

In one embodiment of the present disclosure, the computing device 100 may train a prediction model using a learning dataset containing the determined input variables and adverse reaction information for a specific drug and a specific disease, such that the artificial intelligence-based prediction model outputs adverse reaction information for the specific drug and the specific disease in response to receiving the input variables (S330).

In one embodiment, the method of training the prediction model for adverse drug reaction detection may include determining the prediction model for adverse drug reaction detection among a plurality of candidate models, and the prediction model may be trained to output whether an adverse drug reaction occurs for a specific drug or a specific disease, or a probability of occurrence of the adverse drug reaction for a specific drug or a specific disease, using the preprocessed input variables as input data.

In one embodiment, the plurality of candidate models may be one or more models selected from candidate models consisting of Random Forest, Logistic Regression, Support Vector Machine, Decision Tree, Naive Bayes, k-Nearest Neighbor, XGBoost, LightGBM, CatBoost, Convolutional Neural Network (CNN), Recurrent Neural Network (RNN), Long Short-Term Memory (LSTM), Bidirectional Long Short-Term Memory (BiLSTM), Transformer, and Generative Pre-trained Transformer (GPT).

In one embodiment, the prediction model may obtain output results from each of the candidate models using a predetermined test dataset, and may determine one of the candidate models as the prediction model based on a performance evaluation of the output results from each of the candidate models.

In an additional embodiment, the performance evaluation of the output results of the candidate models may be based on Area Under the Curve (AUC), accuracy, F1-score, precision, recall, sensitivity, and/or specificity.

In one embodiment, the input variables used in training and/or inference of the prediction model may be preprocessed through performing outlier processing, missing value imputation, categorization/re-categorization, and/or normalization/standardization.

In an additional example, large-volume data including medications being taken, possible adverse drug reactions, and patient demographic information may be acquired and updated in real time from public data databases (e.g., FAERS, KAERS, WHO-ART, SIDER, and EU-ADR).

In an additional embodiment, the prediction model according to the present disclosure may output even ADRs with low probability of occurrence as meaningful prediction results for administered drugs and indications having relatively very large amounts of accumulated data, despite the vastness of such data, by considering the target patient's underlying health issues and whether the patient is an infant or child.

In one embodiment, the prediction model may improve learning and/or inference efficiency and/or accuracy through adverse drug reaction prediction results of the prediction model referenced at each epoch performed multiple times. Specifically, such improvements in efficiency and/or accuracy may be achieved by receiving feedback on ground truth data for adverse drug reactions confirmed with clinically high causality through supervised learning, semi-supervised learning, and/or unsupervised learning.

As a specific example, for ADR prediction results when considering administration of aminosalicylic acid (5-ASA) and CAM inhibitor antibody drug (Vedolizumab) as treatment options for ulcerative colitis discovered within 6 months in a 47-year-old male, if the prediction model according to the present disclosure outputs tiredness 34.4%, joint pain 30.2%, headache 28.9%, and bronchitis 19.3%, based on these results, a medical expert may decide to administer the 5-ASA and Vedolizumab for treatment of ulcerative colitis in the patient, and may be provided with suggestions for concomitant medications to prevent such ADRs based on the expected probability of occurrence and severity of the anticipated ADRs.

In an additional embodiment, adverse drug reaction data that occurred or did not occur according to monitoring of treatment progress may be reflected in updating a loss function or hyperparameters of the prediction model that outputs the ADR prediction results. As an example, when bronchitis actually occurs with certain causality at moderate severity in the 47-year-old male ulcerative colitis patient who received this as a prediction result, feeding back this data to the prediction model may be an essential process.

Specifically, in one embodiment, the computing device 100 may predict, according to information included in the periodic conversion database, possible ADRs from drug Z administered to cardiovascular disease patient X for indication Y as follows: i. moderate ADR of inflammatory bowel disease (IBD) with 3.8% occurrence probability, ii. mild ADR of diarrhea with 13.2% occurrence probability, and iii. minor ADR of dyspepsia with 10.9% occurrence probability.

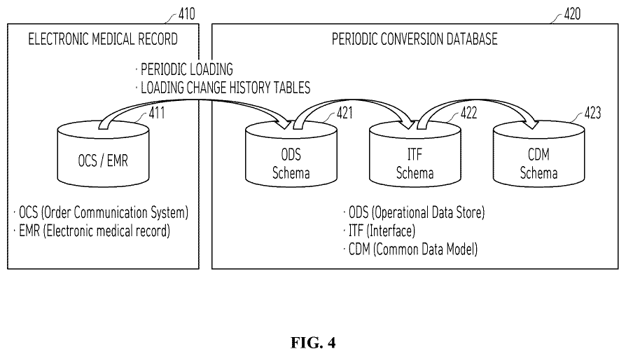

Additional description of the learning and operation of the prediction model will be described later with reference to FIGS. 4 to 7.

FIG. 4 illustrates a flowchart for building a periodic conversion database 420 including principles for periodic data loading and change history table loading of an electronic medical record 410 and a periodic conversion database 420 according to one embodiment of the present disclosure.

In one embodiment, FIG. 4 illustrates a feature in which the computing device 100 periodically loads data and change history of an OCS and/or EMR 411 from an order communication system (OCS) and/or electronic medical record (EMR) 411 to the periodic conversion database 420. In one embodiment, FIG. 4 illustrates that the computing device 100 may store data using an Operational Data Store (ODS) 421, an Interface (ITF) 422, and a Common Data Model (CDM) 423.

In one embodiment, the ODS 421 is an operational data storage, which may refer to a central database that provides a snapshot of latest data from multiple transactional systems for operational reporting. In one embodiment, the ODS 421 may collect or receive information from the OCS and/or EMR 411 that has been newly added or has undergone changes at predetermined time intervals. In one embodiment, the ODS 421 may be used as a database designed to integrate data from multiple sources.

The ODS 421 that receives newly added or changed OCS and/or EMR 411 information may transmit such information through an ITF (422) schema to be stored as a CDM 423 schema. The common data model (CDM) 423 may refer to a data model having the same structure and specifications that may be applied to medical data having different structures possessed by respective medical institutions. The common data model (CDM) 423 may allow the same analysis code to be executed individually at each data-holding institution, thereby enabling distributed collaborative research that integrates distributed data.

In one embodiment according to the present disclosure, the periodic conversion database 420 may store information such as patients'demographic information, drugs and indications administered to patients, adverse drug events (ADEs) that have occurred, and medical history at predetermined time intervals or optimal time intervals determined by the prediction model. The periodic conversion database 420 may refer to a data storage that collects and builds data on newly entered or additionally entered ADR and patient information.

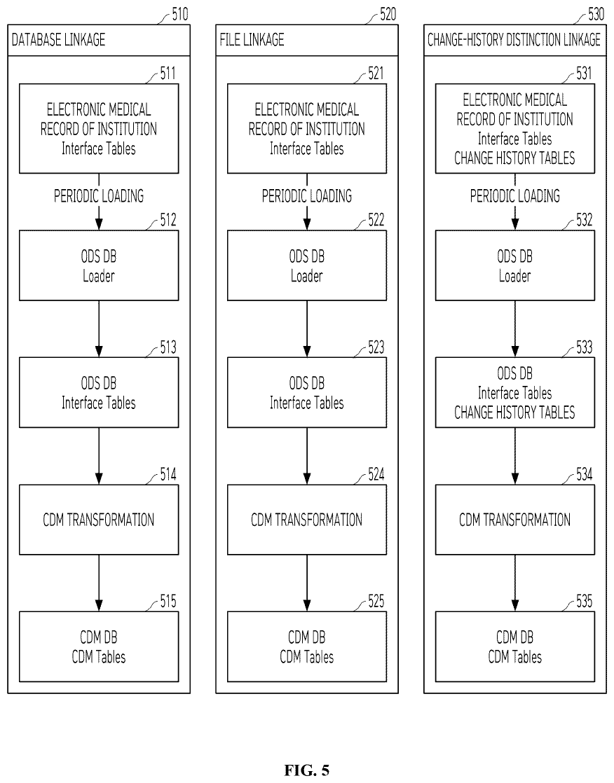

FIG. 5 illustrates a concept of file integration of building a periodic conversion database according to one embodiment of the present disclosure.

In one embodiment, the computing device 100 may build the periodic conversion database 420 using at least one technique among a database linkage (510) technique, a file linkage (520) technique, and/or a change-history distinction linkage (530) technique.

In one embodiment, in connection with the linkage of the database 510, the computing device 100 may be periodically loaded with data from the interface tables 511 where the data is entered in the electronic medical record (EMR) of the medical facility. The loaded data may be passed to ODS DB interface tables 513 through an operational data store database loader 512. The computing device (100) may link the loaded data with the CDM table in the CDM DM by applying a common data model (CDM) transformation 514 to the ODS DB interface tables 513. By the aforementioned scheme, a database related to the EMR and a common data model database may be linked with each other.

The database linkage (510) technique may be utilized in the same or similar manner as the file synchronization (520) technique and/or change-history distinction linkage (530) technique described later.

In one embodiment, the file linkage (520) technique may refer to a data automation method that enables more accurate and/or efficient linkage of disease onset age, duration in months, symptom improvement age, and cure age, for example, based on information about the patient's age at the time of data entry, disease occurrence time, and disease duration. Specifically, with regard to the file linkage 520, the computing device 100 may upload the data to the ODS DB through periodic loading of data from the medical institution's EMR, comma-separated values (CSV) file, or Parquet file 521. Data loaded into the ODS DB interface tables 523 through the ODS DB loader 522 may be uploaded. In one embodiment, EMR-related files loaded into the ODS DB interface tables 523 may be stored in the format of CDM tables 525 of the CDM database through CDM conversion 524.

In one embodiment, the file linkage (520) technique may refer to a method for linking electronic medical record (EMR) documents 521 to a file format most suitable for operating a prediction model and simultaneously operating with an external model different from the prediction model, without loss of information contained therein, even when EMR document (521) extensions differ from each other.

In one embodiment, the change-history distinction linkage (530) technique may refer to a method for organically classifying and linking ADR-related changes according to information such as newly occurring symptoms, disappeared symptoms, symptom severity, added or excluded medications, smoking status, drinking status, and newly discovered medical history for the same patient. In one embodiment, in connection with the change-history distinction linkage 530, the computing device 100 may periodically receive data from interface tables 531 and change history tables 531 where the data is entered in the electronic medical record (EMR) of the medical institution. The computing device 100 may perform upload to a temporary logic-regional DB of data warehouse through an ODS DB loader 532. The computing device 100 may load data to the interface tables and the change history tables 533 of the ODS DB through the ODS DB loader 532. The loaded data may be stored in a CDM database 535 via CDM transformation 534. In one embodiment, since the change classification linkage (530) technique targets and links change data by using change history tables storing the changed data, a computing resource may be efficiently used in data linkage.

In one embodiment according to the present disclosure, the update cycle of the periodic conversion database 420 that is updated using at least one of the database linkage 510 technique, the file linkage 520 technique, or the change-history distinction linkage 530 technique may correspond to a predetermined time period. As an example, the update cycle of the periodic conversion database 420 may be changed from once a month to once a week and/or daily according to user needs. As an example, the update cycle of the periodic conversion database 420 may be determined based on a quantitative amount of computing resources of the computing device 100. For example, the computing device 100 may shorten or extend the update cycle depending on the sufficiency/insufficiency of computing resources.

In one embodiment, the computing device 100 may obtain one or more of demographic data, medication intake related data, disease diagnosis data, diagnostic test data, and nursing record data from a public database (e.g., FAERS, KAERS, WHO-ART, SIDER, EU-ADR, etc.). The public databases are not limited to the databases disclosed herein.

FIG. 6 exemplarily illustrates an overall process including database building, development of artificial intelligence for an adverse drug reaction detection, and notification of an abnormality detection result in sequence according to one embodiment of the present disclosure.

The “adverse drug reaction (ADR)” refers to a harmful and unintended reaction that occurs when a pharmaceutical product or the like is administered or used, where a causal relationship with the pharmaceutical product or the like cannot be excluded, and may include not only adverse reactions occurring at normal doses but also adverse drug reactions and withdrawal symptoms that occur when a drug is intentionally or accidentally used in overdose or when a drug is abused, as well as cases where the expected pharmacological action does not appear.

“Adverse drug reaction monitoring” refers to activities for rapidly and systematically collecting or evaluating various adverse events occurring during pharmaceutical use to implement countermeasures and communicate safety information and action results to medical professionals, consumers, and others, thereby establishing rational pharmaceutical use and preventing drug-related harm in advance.

As a concept related to the ADR, there may be a side effect and an adverse drug event (ADE). The side effect is a concept opposite to a principal action that appears when a pharmaceutical is used for a specific purpose, referring to all unintended effects that occur when a drug is administered according to a normal dosage. Since this the side effect means all actions other than those intended for therapeutic purposes, the side effect is a concept that may be actively utilized in various fields that comprehensively study all actions regardless of the presence or absence of harmfulness.

The adverse drug event (ADE) refers to an undesirable and unintended sign, symptom, or disease that occurs during administration or use of a pharmaceutical product or the like, regardless of a causal relationship with the pharmaceutical product.

The adverse drug reaction (ADR) is a harmful and unintended reaction occurring during administration or use of pharmaceuticals according to the normal dosage, where a causal relationship with the pharmaceutical cannot be excluded, and since the ADR is predictable and preventable in advance, management by pharmacists and medical personnel is considered important.

The drug metabolism occurs mostly in the liver, where a metabolic action takes place in the endoplasmic reticulum of hepatocytes through oxidation, reduction, or hydrolysis reactions of drugs by the cytochrome P450 (CYP450) metabolic enzyme system.

One embodiment in the present disclosure may include a step of deriving predictable ADRs in order of the type of drug administered and genotype/expression type per race/individual, causality (certain/probable/possible/unlikely/conditional) according to anamnesis or categorization (mild/moderate/severe) according to a response degree, and an occurrence frequency.

In one embodiment, the computing device 100 may implement an active drug adverse reaction detection system through the processes of building a periodic conversion database (610), developing drug adverse reaction detection artificial intelligence (620), and notifying abnormality detection results (630).

In one embodiment, the process of building the periodic conversion database (610) may include term standardization and/or term mapping 616 for raw data obtained from an electronic medical record 612 and an adverse drug reaction specification 614. The term mapping 616 may refer to a process of standardizing data formats (e.g., formats) for raw data having various formats. The term mapping 616 may refer to a process of standardizing data representations for raw data having different expressions but the same or similar meanings.

For example, the term mapping (616) process may be operated based on an AI-based language model that determines whether multiple input data correspond to a synonyms or synonymic relationship. In such an example, the artificial intelligence-based language model may extract features of two or more input data that are input, compare those features in a vector space, and based on the results of the comparison, determine whether two or more input data have the synonyms or synonymic relationship with each other. As such, the terms corresponding to the synonyms or synonymic relationship may be managed while being mapped to each other.

In one embodiment, the periodic conversion database 618 may be built according to, for example, OMOP-CDM specifications. In one embodiment, the periodic conversion database 618 may be predetermined to be updated at predetermined time periods (e.g., daily, weekly, monthly and/or yearly intervals).

In one embodiment, the adverse drug reaction detection artificial intelligence development 620 may include a process of modeling and/or validating the prediction model.

In one embodiment, the computing device 100 may perform processes of data preprocessing 622, AI model learning 624, AI model validation 626, and AI model selection 628.

In one embodiment, the data preprocessing 622 may include a process of determining input variables to be used for training the artificial intelligence model among a plurality of term-unified variables in the periodic conversion database. As illustrated in FIG. 6, the data preprocessing 622 may include feature selection, outlier determination, missing value imputation, categorization or re-categorization, and normalization or standardization.

In one embodiment, the processes related to the artificial intelligence model training 624 may include a process of selecting a specific candidate algorithm among multiple candidate algorithms, a process of selecting a specific learning method among multiple learning methods, and a process related to hyperparameter tuning or optimization. As an example, the learning methods may include federated learning and/or incremental learning.

In one embodiment, the processes related to the AI model validation 626 may include a methodology for validation using a test dataset of N:1 and/or a methodology for validation through evaluation from an external server. In one embodiment, the AI model validation 626 may also be performed using performance-related metrics of the AI model, such as AUC, accuracy, F-1 score, etc.

In one embodiment, the computing device 100 may select a specific artificial intelligence model among a plurality of artificial intelligence models (e.g., algorithms) through the artificial intelligence model validation 626.

In one embodiment, the prediction model of the computing device 100 may process data input from the input layer to the hidden layer. Results from residual connections between a plurality of hidden layers may be delivered to the output layer.

According to one embodiment of the present disclosure, the prediction model may include an encoder and a decoder. For example, a first input data set including input variables or combinations of input variables, such as demographic data, is input to the encoder, and the encoder and decoder may be pre-trained such that an output of the decoder that receives an output of the encoder corresponds to the first input data set. Through such pre-training, the encoder may be configured to accurately or efficiently generate features or vectors for the input variables.

According to one embodiment of the present disclosure, the prediction model may include an encoder and a decoder. For example, the pre-training of the prediction model may be performed such that a first input data set including input variables or combinations of input variables such as demographic data is input to the encoder, a second input data set including data on administered drugs and adverse drug reactions is input to the decoder together with the output of the encoder, and the decoder outputs prediction results including administered drug information and possible clinicopathological reactions or occurrence probabilities associated with the ADR.

In one embodiment, the prediction model may categorize each information in the form of embedding and may perform operations required for generating output results according to the assigned categories. As an example, the category embedding of family history information, medical history information, and genetic information may be performed in a separate embedding layer within the prediction model to reflect adverse drug reaction occurrence probability information.

Such an effect according to one embodiment of the present disclosure is that by efficiently and accurately predicting adverse reactions to specific drugs are efficiently and accurately predicted may, for example, in patients having various information, help patients and medical professionals design countermeasures in advance to prevent occurrence of ADRs or minimize impact on vital functions. Further, the computing device 100 may have a structure similar to any arbitrarily assumed specific deep learning network. However, the prediction model in the present disclosure is not limited to the artificial intelligence models and may also be a rule-based operation model.

In one embodiment, in one embodiment for the process for notifying the abnormality detection results 630, the computing device 100 may transmit output results of the prediction model for adverse drug reactions to a user terminal (transceiving device). For example, the output result of the prediction model transmitted to the user terminal may include at least one output result among: a first result indicating binary classification for adverse drug reactions, categorized as detected or undetected; a second result indicating probability notation for adverse drug reactions; or a third result indicating three-level category notation connecting drug, adverse reaction, and risk level. For example, the three-level category notation connecting “drug, ADR, and risk level” output by the prediction model may output a three-level category notation as: single overdose of 10 mg or more bupropion, seizure, and 71.0%, as a severe adverse event.

In one embodiment, the output result from the prediction model may be used to retrain the prediction model based on the accuracy evaluated for the output result. As a specific example, if a 29-year-old male ankylosing spondylitis patient receiving Adalimumab 40 mg once weekly is provided with an output result of 35.2% probability of developing benign, malignant and unidentified neoplasms including polyps as the ADR, and the neoplasm does not actually occur in the patient, the prediction model according to the present disclosure may be provided with such feedback data, i.e., that an unidentified neoplasm did not occur in a 29-year-old male ankylosing spondylitis patient with a specific history upon administration of Adalimumab 40 mg once weekly, as retraining data.

In one embodiment, feedback information corresponding to the output result transmitted from THE user terminal that received the output result from the prediction model may be used to retrain the prediction model. As a specific example, for a patient for whom demographic information and accurate information about medical history are absent, when follow-up data is collected, the follow-up data may be provided to the user terminal that receives the ADR prediction output result from the prediction model, and the corresponding feedback information may be utilized to receive more accurate prediction results from the prediction model or may be used to retrain the prediction model.

Management of the adverse drug reaction has a limitation in that when relying on reporting channels such as adverse drug reaction reporting systems, voluntary reporting by patients and healthcare institutions is required, and if a reporter is not aware of the adverse reactions, the adverse reactions may not be reported, and without timely measures for adverse drug reactions, fatal additional harm may be caused to the patient.

The active adverse drug reaction detection system according to one embodiment of the present disclosure is a system capable of detecting the adverse drug reaction in a timely manner, which not only prevents potential harm that may result from failure to recognize the adverse drug reaction, but also enables simple and systematic management of the adverse drug reaction. The system is particularly efficient in detecting specific drugs for which adverse reactions have been reported in hospitalized patients, but may be used universally without distinction between inpatient/outpatient settings or specific drugs. Therefore, the system may also be usefully employed by institutions with insufficient personnel for managing drug adverse reactions or by individual patients taking medications.

Pharmaceutical companies conduct post-marketing surveillance (PMS) for long-term safety monitoring of drugs, which not only requires significant time, manpower, and costs, but also has major limitations in that subject recruitment and data collection are difficult, requiring active cooperation from medical staffs and patients. The technique according to one embodiment of the present disclosure may reduce a time, manpower, and costs for PMS and further contribute to creating a virtuous cycle structure in the field of drug safety.

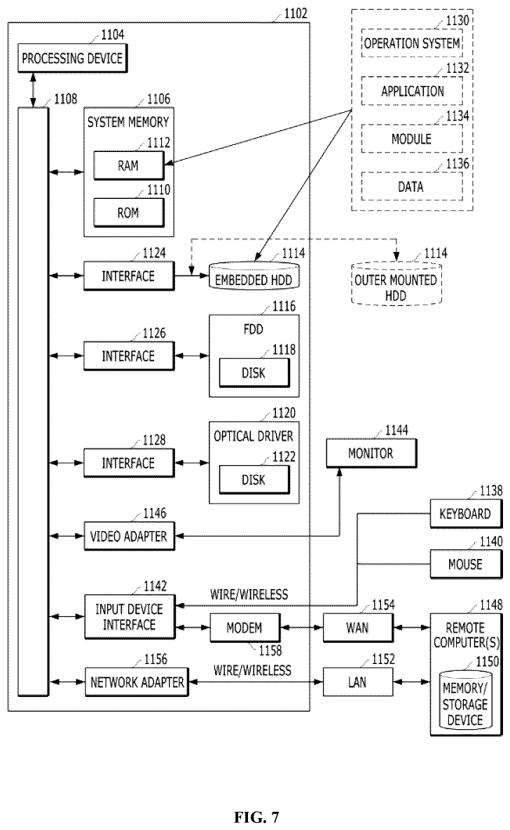

FIG. 7 is a schematic diagram of a computing environment according to one embodiment of the present disclosure.

Components, modules, or units in the present disclosure may include routines, procedures, programs, components, data structures, and the like that perform specific tasks or implement specific abstract data types. In addition, those skilled in the art will readily recognize that the methods presented in the present disclosure may be implemented in other computer system configurations including single-processor or multi-processor computing devices, mini-computers, mainframe computers, as well as personal computers, handheld computing devices, microprocessor-based or programmable consumer electronics, and others (each of which may operate in connection with one or more associated devices).

The embodiments described in the present disclosure may also be implemented in a distributed computing environment where certain tasks are performed by remote processing devices that are connected through a communication network. In such a distributed computing environment, program modules may be located in both local and remote memory storage devices.

A computing device generally includes various computer-readable media. Any media that can be accessed by a computer may be a computer-readable medium, and such computer-readable media include volatile and non-volatile media, transitory and non-transitory media, and removable and non-removable media. As a non-limiting example, the computer-readable media may include both computer-readable storage media and computer-readable transmission media.

The computer-readable storage media include volatile and non-volatile media, transitory and non-transitory media, and removable and non-removable media implemented in any method or technology for storing information such as computer-readable instructions, data structures, program modules, or other data. The computer-readable storage media include, but are not limited to, RAM, ROM, EEPROM, flash memory or other memory technologies, CD-ROM, DVD (digital video disk) or other optical disk storage devices, magnetic cassettes, magnetic tapes, magnetic disk storage devices, or any other medium that can be accessed by the computer and used to store desired information.

The computer-readable transmission media generally embody computer-readable instructions, data structures, program modules, or other data in a modulated data signal such as a carrier wave or other transport mechanism, and include all information delivery media. The term “modulated data signal” means a signal that has one or more of its characteristics set or changed in such a manner as to encode information in the signal. As a non-limiting example, the computer-readable transmission media include wired media such as a wired network or direct-wired connection, and wireless media such as acoustic, RF, infrared, and other wireless media. A combination of any of the above media is also included within the scope of the computer-readable transmission media.

An exemplary environment 2000 implementing various aspects of the present invention including a computer 2002 is illustrated. The computer 2002 includes a processing device 2004, a system memory 2006, and a system bus 2008. As used herein, the term “computer 2002” may be used interchangeably with “computing device.” The system bus 2008 couples system components, including but not limited to the system memory 2006, to the processing device 2004. The processing device 2004 may be any of various commercially available processors. Dual-processor and other multiprocessor architectures may also be used as the processing device 2004.

The system bus 2008 may be any of several types of bus structures, including a memory bus, a peripheral bus, or various commercial bus architectures, and may be additionally connected to a local bus. The system memory 2006 includes a read-only memory (ROM) 2010 and a random access memory (RAM) 2012. A basic input/output system (BIOS), containing basic routines that help transfer information between elements within the computer 2002 during start-up and similar operations, is stored in the non-volatile memory 2010 such as ROM, EPROM, or EEPROM. The RAM 2012 may also include high-speed RAM such as static RAM for caching data.

The computer 2002 further includes an internal hard disk drive (HDD) 2014 (e.g., EIDE, SATA), a magnetic floppy disk drive (FDD) 2016 (e.g., for reading from or writing to a removable diskette 2018), a solid-state drive (SSD), and an optical disk drive 2020 (e.g., for reading from or writing to a CD-ROM disk 2022 or other high-capacity optical media such as a DVD). The hard disk drive 2014, magnetic disk drive 2016, and optical disk drive 2020 can each be connected to the system bus 2008 via a hard disk drive interface 2024, a magnetic disk drive interface 2026, and an optical drive interface 2028, respectively. The interface 2024 for external drive implementations may include at least one of, or both, USB (Universal Serial Bus) and IEEE 1394 interface technologies.

These drives and the computer-readable media associated therewith provide non-volatile storage of data, data structures, computer-executable instructions, and the like. In the case of the computer 2002, the drives and the media correspond to the storage of data in an appropriate digital format. Although the above description of computer-readable storage media mentions mobile optical media such as HDDs, removable magnetic disks, and CDs or DVDs, those skilled in the art will readily understand that other types of computer-readable storage media, such as zip drives, magnetic cassettes, flash memory cards, cartridges, and the like, may also be used in an exemplary operating environment, and any such media may include computer-executable instructions for performing the methods of the present invention.

Multiple program modules, including an operating system 2030, one or more application programs 2032, other program modules 2034, and program data 2036, may be stored in the drives and in the RAM 2012. All or portions of the operating system, applications, modules, and/or data may also be cached in the RAM 2012. It will be well appreciated that the present invention may be implemented in commercially available operating systems or combinations of operating systems.

A user may enter commands and information into the computer 2002 through one or more wired or wireless input devices such as a keyboard 2038 and a pointing device 2040 (for example, a mouse). Other input devices (not shown) may include a microphone, IR remote control, joystick, game pad, stylus pen, touch screen, and the like. These and other input devices are often connected to the processing device 2004 through an input device interface 2042 coupled to the system bus 2008, but may also be connected by other interfaces, including a parallel port, IEEE 1394 serial port, game port, USB port, IR interface, and others.

A monitor 2044 or other type of display device is also connected to the system bus 2008 via an interface such as a video adapter 2046. In addition to the monitor 2044, the computer generally includes other peripheral output devices such as speakers, printers, and the like (not shown).

The computer 2002 may operate in a networked environment using logical connections to one or more remote computers 2048 through wired and/or wireless communication. The remote computer(s) 2048 may be a workstation, server computer, router, personal computer, portable computer, microprocessor-based entertainment apparatus, peer device, or other general network node, and typically include many or all of the elements described relative to the computer 2002, although only a memory storage device 2050 is illustrated for brevity. The logical connections shown include wired and/or wireless connections to a local area network (LAN) 2052 and/or a larger network such as a wide area network (WAN) 2054. Such LAN and WAN networking environments are common in offices and enterprises, and facilitate enterprise-wide computer networks such as intranets, all of which may connect to global computer networks such as the Internet.

When used in a LAN networking environment, the computer 2002 is connected to the local network 2052 through a wired and/or wireless network interface or adapter 2056. The adapter 2056 may facilitate wired or wireless communication to the LAN 2052, which may also include a wireless access point configured to communicate with the wireless adapter 2056. When used in a WAN networking environment, the computer 2002 may include a modem 2058, be connected to a communication server on the WAN 2054, or have other means for establishing communications over the WAN 2054, such as the Internet. The modem 2058, which may be an internal or external, wired or wireless device, is connected to the system bus 2008 via the serial port interface 2042. In a networked environment, the program modules described with respect to the computer 2002, or portions thereof, may be stored in the remote memory/storage device 2050. It will be appreciated that the network connections shown are exemplary and that other means of establishing a communication link between computers may be used.

The computer 1602 operates to communicate with various wireless devices or entities that are disposed and operated through wireless communication, such as printers, scanners, desktop and/or portable computers, PDAs (portable data assistants), communication satellites, devices or places associated with wireless-detectable tags, and telephones. This includes at least wireless fidelity (Wi-Fi) and Bluetooth wireless technologies. Accordingly, such communication may be within a predefined structure such as a conventional network, or may simply be ad hoc communication between at least two devices.

It should be understood that a specific order or hierarchical structure of steps in the presented processes represents one example of exemplary approaches. Based on design priorities, the specific order or hierarchical structure of the steps in the processes may be rearranged within the scope of the present disclosure. The method claims of the present disclosure provide elements of various steps in a sample order, but are not limited to the specific order or hierarchical structure presented herein.

### Mode for Invention

Related contents in the best mode for carrying out the present disclosure are described as above.

### INDUSTRIAL APPLICABILITY

It may be used in a device and a system for managing patients exhibiting adverse reactions to a drug.

1. A method for generating a prediction result for an adverse drug reaction using artificial intelligence technology performed by a computing device, the method comprising:
building a periodically updated transformed database scheduled to be updated at predetermined time intervals using raw data for detecting an adverse drug reaction;
determining a plurality of input variables for training an artificial intelligence-based prediction model for the adverse drug reaction detection based on data stored in the periodically updated transformed database; and
training the prediction model using the determined input variables and a training dataset including adverse reaction information for a specific drug and a specific disease, such that the prediction model outputs the adverse reaction information for the specific drug and the specific disease in response to receiving the input variables,
wherein the step of building the periodically updated transformed database comprises:
converting terminology of the raw data to correspond to a common data standard specification of the prediction model; and
building the periodically updated transformed database scheduled to be updated at the predetermined time intervals using at least one of a database linkage technique, a file linkage technique, or a change-history distinction linkage technique, and
wherein the input variables are determined using a variable importance determination technique that utilizes perturbed input data in which at least a portion of the variables are changed and output data output from the prediction model in response to the perturbed input data.

2. The method of claim 1,
wherein the step of building the periodically updated transformed database further comprises:
acquiring the raw data for detecting an adverse drug reaction from electronic medical records (EMR), wherein the raw data including at least one of medication intake related data, disease diagnosis data, diagnostic test data, or patient nursing record data.

3. The method of claim 1, wherein the raw data is additionally acquired from at least one of FAERS, KAERS, WHO-ART, SIDER, or EU-ADR.

4. The method of claim 1, wherein the plurality of input variables includes at least one of demographic variables, medication intake related variables, diagnostic test related variables, nursing record related variables, or adverse drug reaction diagnosis related variables.

5. The method of claim 4, wherein the demographic variables include at least one of gender, age, or region,
the medication intake related variables include at least one of drug type, medication period, medication dosage, or medication history,
the diagnostic test related variables include at least one of imaging tests, blood tests, or urine tests,
the nursing record-related variables include variables obtained from nursing records using at least one technique of optical character recognition (OCR), natural language processing (NLP), or text mining, and
the adverse drug reaction diagnosis-related variables include information related to a disease diagnosis caused by an adverse drug reaction.

6. The method of claim 1, wherein the input variables are preprocessed based on:
performing outlier processing;
performing missing value imputation;
performing categorization or re-categorization; and
performing normalization or standardization.

7. The method of claim 6, wherein the step of training the prediction model for detecting the adverse drug reaction comprises:
determining the prediction model for detecting the adverse drug reaction among a plurality of candidate models; and
training the prediction model such that, using the preprocessed input variables as input data, the prediction model outputs whether an adverse drug reaction occurs for a specific drug or a specific disease, or a probability of occurrence of the adverse drug reaction for the specific drug or the specific disease.

8. The method of claim 1, wherein the prediction model is a model selected from among candidate models including Random Forest, Logistic Regression, Support Vector Machine, Decision Tree, Naive Bayes, k-Nearest Neighbor, XGBoost, LightGBM, CatBoost, Convolutional Neural Network (CNN), Recurrent Neural Network (RNN), Long Short-Term Memory (LSTM), Transformer, and Generative Pre-trained Transformer (GPT).

9. The method of claim 8, wherein the prediction model is determined based on:
obtaining output results from each of the candidate models using a predetermined test dataset;
performing performance evaluation on the output results from each of the candidate models; and
determining one model among the candidate models as the prediction model based on a result of the performance evaluation.

10. The method of claim 1, wherein the adverse reaction information for the specific drug and the specific disease includes:
first information indicating whether an adverse drug reaction occurs for a specific drug;
second information indicating a probability of occurrence of an adverse drug reaction for the specific drug;
third information indicating whether an adverse drug reaction occurs for a specific disease; and
fourth information indicating a probability of occurrence of an adverse drug reaction for the specific disease.

11. The method of claim 1, further comprising:
transmitting an output result of the prediction model for the adverse drug reaction to a user terminal.

12. The method of claim 11, wherein the output result transmitted to the user terminal includes at least one of:
a first result indicating a binary classification of an adverse drug reaction as detected or not detected;
a second result indicating a probability notation for the adverse drug reaction; or
a third result indicating a three-level categorical notation linking a drug, an adverse reaction, and a risk level.

13. The method of claim 1, wherein an output result output from the prediction model is used to retrain the prediction model based on an accuracy evaluation performed on the output result.

14. The method of claim 1, wherein feedback information corresponding to the output result transmitted from a user terminal that has received the output result of the prediction model is used to retrain the prediction model.

15. (canceled)

16. A computing device comprising:
at least one processor; and
a memory,
wherein the at least one processor is configured to:
build a periodically updated transformed database scheduled to be updated at predetermined time intervals using raw data for detecting an adverse drug reaction;
determine a plurality of input variables for training an artificial intelligence-based prediction model for the adverse drug reaction detection based on data stored in the periodically updated transformed database; and
train the prediction model using the determined input variables and a training dataset including adverse reaction information for a specific drug and a specific disease, such that the prediction model outputs the adverse reaction information for the specific drug and the specific disease in response to receiving the input variables,
wherein the building operation of the periodically updated transformed database comprises:
converting terminology of the raw data to correspond to a common data standard specification of the prediction model; and
building the periodically updated transformed database scheduled to be updated at the predetermined time intervals using at least one of a database linkage technique, a file linkage technique, or a change-history distinction linkage technique, and
wherein the input variables are determined using a variable importance determination technique that utilizes perturbed input data in which at least a portion of the variables are changed and output data output from the prediction model in response to the perturbed input data.

17. A method for generating a prediction result for an adverse drug reaction using artificial intelligence technology performed by a computing device, the method comprising:
acquiring input data for detecting an adverse drug reaction based on data stored in a periodically updated transformed database scheduled to be updated at predetermined time intervals using raw data for detecting an adverse drug reaction; and
acquiring adverse reaction information for a specific drug and a specific disease from the input data using an artificial intelligence-based prediction model, wherein the periodically updated transformed database is generated based on:
converting terminology of the raw data to correspond to a common data standard specification of the prediction model; and
building the periodically updated transformed database scheduled to be updated at the predetermined time intervals using at least one of a database linkage technique, a file linkage technique, or a change-history distinction linkage technique, and
wherein a plurality of input variables for training the prediction model are determined using a variable importance determination technique that utilizes perturbed input data in which at least a portion of the variables are changed and output data output from the prediction model in response to the perturbed input data.
